(19) United States
(12) Patent Application Publication
(10) Pub. No.: US 2012/0109203 A1
(43) Pub. Date: May 3, 2012

(54) SPINOUS PROCESS IMPLANT WITH EXTENDED POST

(75) Inventors: Randall F. Dryer, Austin, TX (US); Greg C. Marik, Collierville, TN (US); Charles Schulte Sullivan, III, Germantown, TN (US); Steven D. DeRidder, Bartlett, TN (US)

(73) Assignee: WARSAW ORTHOPEDIC, INC., Warsaw, IN (US)

(21) Appl. No.: 12/916,745

(22) Filed: Nov. 1, 2010

(51) Int. Cl. A61B 17/70; A61B 17/88

(52) U.S. Cl. 606/249; 606/279

(57) ABSTRACT

Spinous process implants that may include a first plate sized to extend along a first lateral side of the spinous processes and a second plate sized to extend along a second lateral side of the spinous processes. The second plate may include a bore that extends through the second plate. An elongated post may extend through the bore with a first end attached to the first plate and a second end positioned outward beyond the outer surface of the second plate away from the first plate. A joint may be positioned along the post that attaches a first section of the post to a second section of the post. The joint may be constructed to align the first and second sections in a collinear orientation and to remove the second section from the first section. Additional joints may also be positioned along the post. The bore may be sized relative to the post for the second plate to be movable along a length of the post.

### BACKGROUND

The present application is directed to devices and methods for stabilizing vertebral members, and more particularly, to interspinous implants to engage onto the spinous processes.

Vertebral members comprise a body, pedicles, laminae, and processes. The body includes sections on inferior and superior ends and has an hourglass shape with a thinner middle section and wider ends. Intervertebral discs are positioned between the bodies of adjacent vertebral members to permit flexion, extension, lateral bending, and rotation. The pedicles are two short rounded members that extend posteriorly from the body, and the laminae are two flattened members that extend medially from the pedicles. The processes are projections that serve as connection points for the ligaments and tendons. The processes include the articular processes, transverse processes, and the spinous process. Each vertebral member has four articular processes including two superior and two inferior processes. The superior processes of a first vertebral member join with the inferior processes of a second vertebral member to form facet joints. The facet joints work with the intervertebral discs to allow motion of the spine. The spinous process is a single member that extends posteriorly from the junction of the two lamina. The spinous process may act as a lever to effect motion of the vertebral member.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion.

One manner of correcting the damage is insertion of an implant onto the spinous processes. The implant may reduce or eliminate the pain and neurological deficit.

### SUMMARY

The present application is directed to implants for attaching to spinous processes. The implants may include a first plate sized to extend along a first lateral side of the spinous processes and a second plate sized to extend along a second lateral side of the spinous processes. The second plate may include a bore that extends through the second plate. An elongated post may extend through the bore with a first end attached to the first plate and a second end positioned outward beyond the outer surface of the second plate away from the first plate. A joint may be positioned along the post that attaches a first section of the post to a second section of the post. The joint may be constructed to align the first and second sections in a collinear orientation and to remove the second section from the first section. Additional joints may also be positioned along the post. The bore may be sized relative to the post for the second plate to be movable along a length of the post.

The present application is also directed to methods of attaching an implant to spinous processes. The methods may include positioning a first plate on a first lateral side of the spinous processes and positioning a post that extends outward from the first plate through an interspinous space formed between the spinous processes. The method may include positioning a second plate on a second lateral side of the spinous processes with the post extending through a bore in the second plate. The second plate may be slid along the post towards the spinous processes from a distal section of the post, over a joint, and onto a proximal section of the post. The first plate may be positioned against the first lateral side of the spinous processes and the second plate against the second lateral side of the spinous processes. The second plate may be secured on the proximal section of the post with the first and second plates contacting against the lateral sides of the spinous processes. The distal section may be detached from the proximal section of the post at the joint.

The various aspects of the various embodiments may be used alone or in any combination, as is desired.

### BRIEF DESCRIPTION OF THE DRAWINGS

FIG. 1 is a side view of an implant mounted to spinous processes according to one embodiment.

FIG. 2 is a perspective view of an implant according to one embodiment.

FIG. 3 is a section view cut along line III-III of FIG. 2 of the post mounted to the plate.

FIG. 4 is a section view similar to FIG. 3 of a post mounted to a plate according to one embodiment.

FIG. 5 is a section view cut along line V-V of FIG. 2 of the locking element engaging the post.

FIG. 6 is a side view of a post according to one embodiment.

FIG. 7A is a sectional view cut along line VII-VII of FIG. 1 according to one embodiment.

FIG. 7B is a sectional view cut along line VII-VII of FIG. 1 according to one embodiment.

FIG. 7C is a sectional view cut along line VII-VII of FIG. 1 according to one embodiment.

FIG. 8A is a section view cut along line VIII-VIII of FIG. 6 according to one embodiment.

FIG. 8B is a section view cut along line VIII-VIII of FIG. 6 according to one embodiment.

FIG. 9 is a section view cut along a longitudinal axis of a post illustrating portions of first and second sections and a joint according to one embodiment.

FIG. 10 is a section view cut along a longitudinal axis of a post illustrating portions of first and second sections and a joint according to one embodiment.

FIG. 11 is a sectional view cut along a longitudinal axis of a post illustrating portions of first and second sections and a joint according to one embodiment.

FIG. 12 is a perspective view of a first section of a post separated from a second section of a post at a joint according to one embodiment.

FIG. 13 is a perspective view of a first section of a post separated from a second section of a post at a joint according to one embodiment.

FIG. 14 is a side view of a second section of a post attached to a first section of a post according tone embodiment.

FIG. 15 is a side view of a post according to one embodiment.

FIG. 16 is a side view of a post according to one embodiment.

### DETAILED DESCRIPTION

The present application is directed to a spinous process implant with two plates that are connected together with a post. The implant is configured for each plate to be positioned on outer lateral sides of spinous processes with the post extending through the interspinous space. One or both plates may be movable along the length of the post to accommodate different anatomies such as for relatively wide or thin spinous processes. The post includes an adequate length for the plates to remain on the post and prevent disengagement. The post also includes a joint to remove a distal section. The distal section is usually removed after the implant is adjusted and secured to the spinous processes. The post may also include additional joints to remove different lengths of the post.

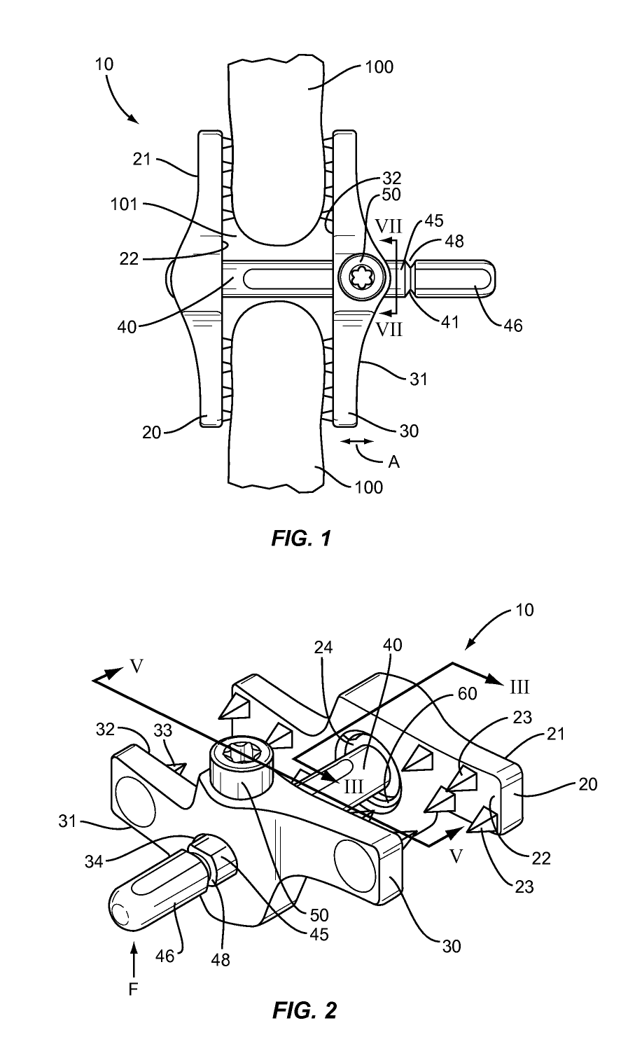

FIG. 1 is a rear view of the implant 10 secured to adjacent spinous processes 100. The implant 10 includes first and second plates 20, 30 secured to opposing outer lateral sides of the spinous processes 100. A post 40 connects the plates 20, 30 together and extends through the interspinous space 101 between the spinous processes 100. The second plate 30 can slide along the length of the post 40 to adjust a distance between the plates 20, 30 to accommodate the spinous processes 100. A locking element 50 secures the plate 30 to the post 40 at the desired position. The post 40 includes a joint to remove a section 46 after the implant 10 is secured to the spinous processes 100.

FIG. 2 illustrates the implant 10 prior to attachment to the spinous processes 100. The first plate 20 includes an outer side 21 that faces away from the spinous processes 100 when attached in a patient, and an inner side 22 that faces towards the spinous processes 100. Likewise, the second plate 30 includes an outer side 31 and an inner side 32. One or both plates 20, 30 may include teeth 23, 33 respectively that extend outward from the inner sides 22, 32. The teeth 23, 33 may extend along an entirety or a limited section of the plates 20, 30. The teeth 23, 33 may include a sharpened tip to engage with the spinous processes 100 to securely position the implant 10. The plates 20, 30 may include the same or different shapes and sizes. FIGS. 1 and 2 include embodiments with the plates 20, 30 having substantially the same shape and size.

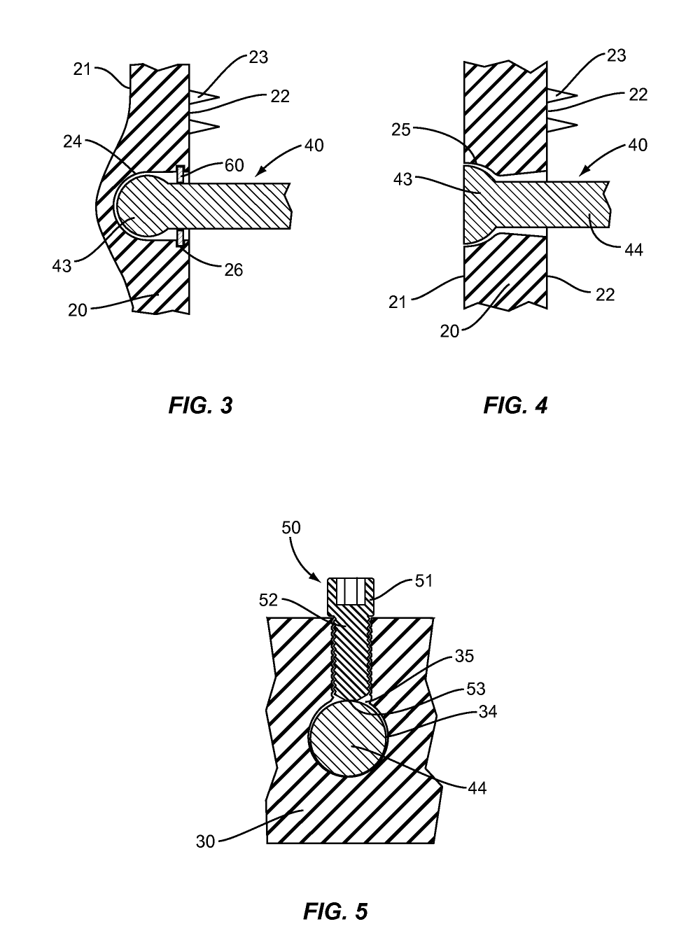

The first plate 20 includes a receptacle 24 to receive a head 43 of the post 40. FIG. 3 includes the receptacle 24 extending inward a limited distance from the inner side 22 and being sized to receive the head 43. The head 43 and receptacle 24 may be sized and shaped for the post 40 to pivot relative to the plate 20. FIG. 3 includes each of the head 23 and receptacle 24 having a rounded sectional shape for the post 40 to pivot within the plate 20.

A locking member 60 may be attached to the plate 20 to secure the head 43 within the receptacle 24. In one embodiment, the locking member 60 is positioned within a groove 26 in the plate 20. Other embodiments include the locking member 60 secured to the plate 20 by mechanical fasteners or adhesives. The locking member 60 includes an opening for the post 40 to extend outward from the receptacle 24. In one embodiment, the locking member 60 is a C-ring that fits within the groove 26.

The receptacle 24 may also extend completely through the first plate 20 as illustrated in FIG. 4. The receptacle 24 may include an enlarged recess 25 in the outer side 21 to receive the head 43 of the post 40. The depth of the recess 24 may allow for the head 43 to be positioned flush or recessed inward from the outer side 21. The shape of the post 40 and the receptacle 24 may allow for pivoting movement of the post 40 relative to the plate 20. In another embodiment (not illustrated), the first plate 20 and post 40 are constructed as a single piece. This may include the plate 20 and post 40 formed as a single unitary piece.

The second plate 30 includes a bore 34 to receive the post 40. The bore 34 is sized relative to the post 40 for the plate 30 to be movable along the length of the post 40 for adjusting a distance from the first plate 20. The bore 34 may include various cross-sectional shapes and sizes depending upon the post 40.

As illustrated in FIG. 5, the plate 30 may also include a cavity 35 that extends inward from an exterior surface and intersects with the bore 34. The cavity 35 extends into the plate 30 at a transverse angle relative to the bore 34. In one embodiment, a longitudinal axis of the cavity 35 is perpendicular to a longitudinal axis of the bore 34. The locking element 50 is configured to fit within the bore 34 and engage the post 40. The locking element 50 includes a head 51 and an outwardly-extending shaft 52. The shaft 52 and cavity 35 may each be threaded to facilitate the positioning of the locking element 50 within the plate 30 and against the post 40. The shaft 52 further includes a tip 53 that contacts against the post 40. The tip 53 may be substantially flat, or may include a curved shape to increase an amount of contact with the post 40. The tip 53 may also include teeth or other like surface configurations to further facilitate the contact with the post 40. The shaft 40 may include a flat section that faces towards the cavity 35 to facilitate the engagement with the tip 53.

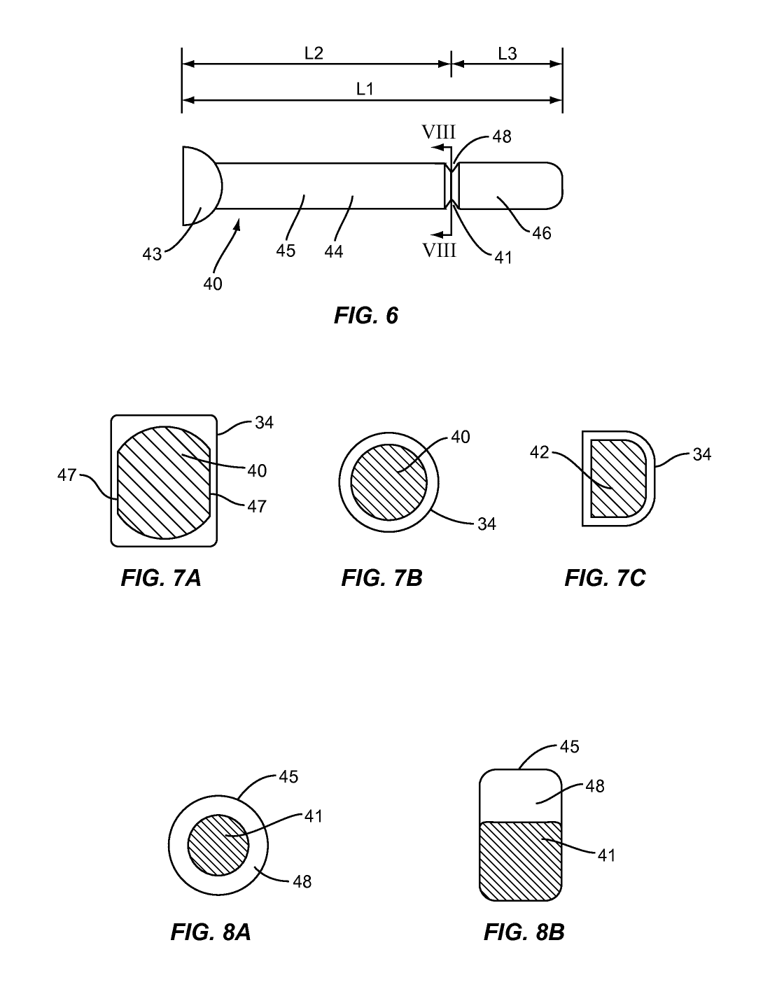

The post 40 includes a head 43 and a shaft 44 as illustrated in FIG. 6. The post 40 includes an overall length L1 defined between the first and second ends to position the plates 20, 30 various distances apart to accommodate various sizes of spinous processes 100. The post 40 further includes a first section 45 with a length L2 that extends between the first end at the head 43 and the joint 41, and a second section 46 that extends between the second end at the tip and the joint 41 and having a length L3. The first section 45 remains within the patient after the second section 46 is removed from the patient. The first and second sections 45, 46 may be collinear as illustrated in FIG. 6.

The post 40 and the bore 34 may include various sectional shapes to control the rotational orientation of the second plate 30 relative to the post 40. FIG. 7A includes the first section 40 with a pair of flat sides 47 that align with flat sides of the bore 34 to prevent the plate 30 from rotating relative to the post 40. The cross-sectional sizes of the post 40 and bore 34 allow for movement of the plate 30 along the length of the post 40. Other shapes may also be provided that allow for movement along the length but prevent or limit rotational movement. FIG. 7B illustrates an embodiment with a circular sectional shape that may be used with a plate 30 having a bore 34 of a similar shape to allow for rotation of the plate 30 about the post 40. FIG. 7C illustrates and embodiment with an asymmetrical post 40 and bore 34 to control an orientation of the second plate 30 relative to the post 40.

The first and second sections 45, 46 may include the same or different sectional shapes. In one embodiment, the shapes are the same.

The joint 41 is positioned at an intermediate point along the shaft 44 between the first and second ends. As illustrated in FIG. 6, the joint 41 may be positioned in closer proximity to the second end at the tip of the post 40 than to the first end at the head 43. The joint 41 secures the second section 46 to the first section 45 to allow the plate 30 to move along the length of the post 40 during the insertion of the implant 10 into the patient. The joint 41 may further be constructed to position the first and second sections 45, 46 in a collinear orientation. The joint 41 may include a variety of constructions, including a weakened strength and a mechanical connection.

The joint 41 may be weaker than the first and second sections 45, 46 and fracture upon the application of an external force on the second section 46. The external force may be substantially perpendicular to a longitudinal axis of the post 40 as illustrated by arrow F in FIG. 2. The external force may also be a torsional force applied to the second section 46.

One construction for a weakened joint 41 includes a notch 48 that extends around a portion or the entirety of the periphery of the shaft 44. FIGS. 1 and 2 include the notch 48 extending around the entire periphery of the post 44. The notch 48 causes the joint 41 to have a smaller cross-sectional area than the adjacent first and second sections 45, 46 causing a fracture to occur at the joint 41. FIG. 8A illustrates a notched joint 41 with a smaller cross-sectional area than the adjacent first section 45. The notch 48 extends around the periphery of the post in this embodiment. FIG. 8B includes a notched joint 41 with the notch 48 extending around a limited portion of the periphery.

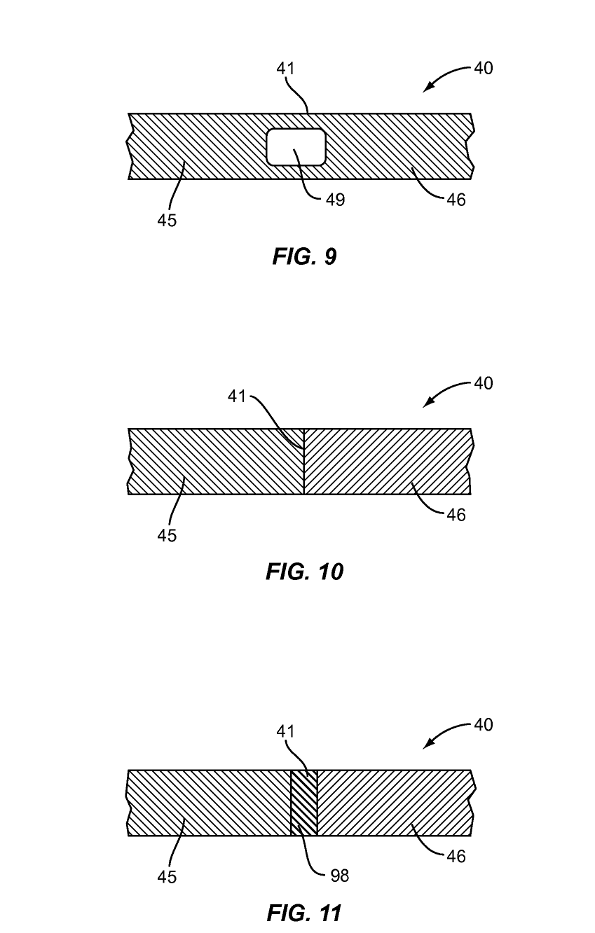

FIG. 9 includes the weakened joint 41 formed by a void 49 in the interior of the post 40. The void 49 weakens the joint 41 thus causing a fracture when an external force is applied to the post 40. FIG. 9 includes the joint 41 having a single void 49, although other embodiments may include joints 41 with multiple voids 49.

The weakened joint 41 may also be formed by the post 40 having an abutment face between different materials. FIG. 10 includes the first and second sections 45, 46 constructed from different materials and secured together at the joint 41. This joint 41 is weaker than the first and second sections 45, 46 thus causing a fracture upon the application of an external force. The sections 45, 46 may be secured together by various techniques, including adhesives.

FIG. 11 includes the joint 41 formed by a weaker material 98 than that of the first and second section 45, 46. The fracture may occur at the connection between the material 98 and one of the sections 45, 46, or may occur within the material 98 causing portions of the material 98 to remain attached to each of the sections 45, 46 after the fracture.

The different aspects that form the weakened joint 41 may be used separately or in combination. For example, the joint 41 may include a notch 48 and a void 49, and a joint 41 with a notch 48 positioned at a connection between first and second sections 45, 46 that are constructed of different materials.

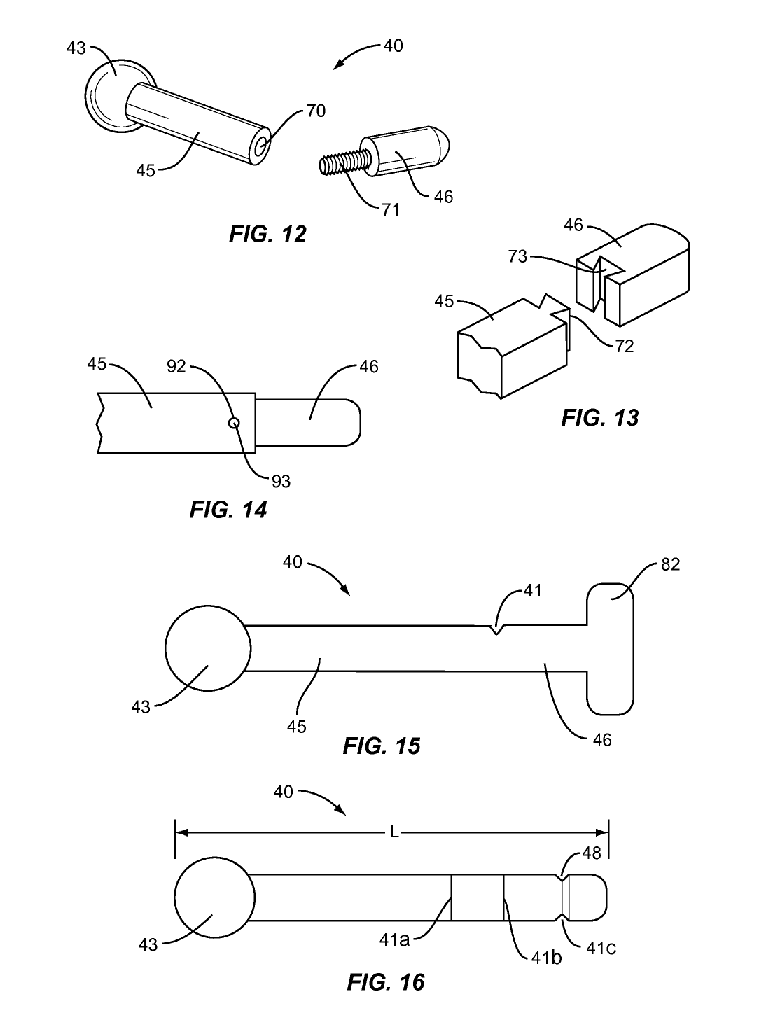

The joint 41 may also include a mechanical connection that secures together the first and second sections 45, 46. FIG. 12 includes a mechanical connection featuring a receptacle 70 in the first section 45 that receives a post 71 that extends outward from the second section 46. Each of the receptacle 70 and post 71 may be threaded to facilitate removal of the second section 46. FIG. 12 includes the aperture 70 positioned in the first section 45 and the post 71 extending from the second section 46. The orientation may also be reversed with the first section 45 including the post 71 and the second section 46 including the aperture 70.

Another mechanical connection is illustrated in FIG. 13 and includes an extension 72 on the first section 45 and a corresponding receptacle 73 on the second section 46. The extension 72 is sized to fit within the receptacle 73 to attach the sections 45, 46 together. In this embodiment, the extension 72 and receptacle 73 each include a dovetailed configuration. Removal of the second section 46 requires applying a force and sliding the second section 46 out of attachment with the first section 45. A ball-and-detent feature may also be included to further secure the sections 45, 46. Removal of the second section 46 occurs with a force applied substantially perpendicular to the longitudinal axis of the post 40 causing the second section 46 to slide out of the first section 45. FIG. 13 includes the extension 72 on the first section 45 and the receptacle 73 on the second section 46. The orientation may also be reversed.

FIG. 14 includes an embodiment with the second section 46 overlapping in a telescoping arrangement with the distal end of the first section 45. The sections 45, 46 each include apertures 92 that align together and receive a fastener 93 to secure the sections 45, 46 together. The fastener 93 is removed from the apertures 92 for removal of the second section 46.

In use, the implant 10 is positioned in the patient with the first plate 20 on a first lateral side of the spinous processes 100 and the second plate 30 positioned on an opposing second lateral side of the spinous processes 100. The post 40 extends outward from the first plate 20 and through the interspinous space 101 and through the bore 34 in the second plate 30. In one embodiment, the second plate 30 is positioned on the post 40 prior to insertion into the patient (i.e., the complete implant 10 of the plates 20, 30 and post 40 are inserted as a single unit into the patient). In another embodiment, the implant 10 may be inserted into the patient as separate elements and attached together within the patient.

The extended length of the post 40 that includes the first and second sections 45, 46 allows the plates 20, 30 to be separated apart by a large amount to facilitate the positioning relative to the spinous processes 100. The distance between the plates 20, 30 can be increased well beyond what is necessary to position each of the plates 20, 30 on the opposing lateral sides of the spinous processes 100 and maintain the plates 20, 30 on the post 40. The enlarged distance between the plates 20, 30 positions the second plate 30 on the second section 46 of the post 40.

Once positioned, the plates 20, 30 are moved together and into contact with the lateral sides of the spinous processes 100. This includes the second plate 30 moving along the length of the post 40 from the second section 46, over the joint 41, and onto the first section 45. The plates 20, 30 may be moved together to apply a compressive force to the spinous processes 100. Once positioned the proper distance apart, the locking element 50 is engaged with the post 40 to maintain the spacing of the plates 20, 30.

With the plate 30 secured to the first section 45 of the post 40, the second section 46 is no longer necessary. The second section 46 is removed by fracturing the joint. Once separated, the second section 46 can be removed from the patient.

FIG. 15 includes an embodiment with the post 40 having a boss 82 along the second section 46. The boss 82 includes a larger cross-sectional size than the bore 34 and prevents the second plate 30 from moving beyond the second section 46 of the post 40. The boss 82 may be positioned at various locations along the length of the second section 46, including the distal end as illustrated in FIG. 15, or in closer proximity to the joint 41. The boss 82 may also provide a gripping surface to apply the force to the post 40 to remove the second section 46.

The second section 46 may include a longitudinal section with a different cross-sectional shape to form one or more gripping surfaces for applying the force. In one embodiment, a majority of the second section 46 includes a circular cross-sectional shape with the gripping section including opposing flattened sides. The flattened sides facilitate contact with the second section 46 and application of the force.

The post 40 may include more than one joint 41 located along the longitudinal length L. The different joints 41 provide for separating different lengths of the post 40 from the remainder depending upon the patient anatomy and the needs of the particular surgical procedure. FIG. 16 includes an embodiment with three joints 41a, 41b, 41c located along the length of the post 40. The multiple joints 41 provides for a single implant 10 to be used for a variety of different surgical procedures on patients with different anatomies and the post 40 can be manipulated during the surgical procedure as necessary.

The multiple joints 41 may be evenly spaced apart along the length of the post 40. Alternatively, the joints 41 may be spaced at different, non-equal intervals. Further, the number of joints 41 may vary.

The various joints 41 along the length L of the post 40 may have the same or different constructions. FIG. 16 includes an embodiment with joints 41a, 41b having a weakened construction formed by the attachment of different materials, and joint 41c including a notch 48 that extends around the circumference of the post 40. The various joints 41 may include a variety of configurations as explained above.

A spacer may also be positioned around the post 40 between the first and second plates 20, 30. The spacer includes a central bore that receives the post 40. The spacer includes a first contact surface to contact against the first spinous process, and a second contact surface to contact against the second spinous process.

U.S. patent application Ser. No. 12/916,761 entitled Spinous Process Implant with a Post and an Enlarged Boss, filed on the same day as the present application, discloses an interspinous device with a pair of plates and a post, and is herein incorporated by reference in its entirety.

The implants 10 may be implanted within a living patient for the treatment of various spinal disorders. The implant 10 may also be implanted in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An implant for attaching to spinous processes comprising:
a first plate sized to extend along a first lateral side of the spinous processes;
a second plate sized to extend along a second lateral side of the spinous processes, the second plate including a bore that extends through the second plate between an inner surface that faces towards the spinous processes and an opposing outer surface;
an elongated post that extends through the bore with a first end attached to the first plate and a second end positioned outward beyond the outer surface of the second plate away from the first plate;
a joint positioned along the post that attaches a first section of the post that extends between the first end and the joint to a second section of the post that extends between the second end and the break section, the joint constructed to align the first and second sections in a collinear orientation and to remove the second section from the first section;
the bore sized relative to the post for the second plate to be movable along a length of the post.

2. The implant of claim 1, wherein the joint includes a notch that extends into the post with the joint having a smaller cross-sectional area than the adjacent first and second sections.

3. The implant of claim 1, wherein the joint includes a void positioned within an interior of the joint.

4. The implant of claim 1, wherein the joint includes an abutment face between the first section constructed from a first material and the second section constructed from a different second material.

5. The implant of claim 1, wherein the joint is constructed from a different material than each of the first and second sections.

6. The implant of claim 1, wherein the joint includes a post that extends outward from one of the first and second sections and fits within an aperture formed in the other of the first and second sections.

7. The implant of claim 1, further comprising at least one additional joint located along the post and being spaced along a longitudinal length of the post away from the joint.

8. An implant for attaching to spinous processes comprising:
a first plate sized to extend along a first lateral side of the spinous processes;
a second plate sized to extend along a second lateral side of the spinous processes, the second plate including a bore that extends through the second plate between an inner surface that faces towards the spinous processes and an opposing outer surface;
an elongated post that extends through the bore with a first end positioned at the first plate and a second end positioned outward beyond the outer surface of the second plate away from the first plate, the post sized to fit in the bore for the second plate to be movable along the post; and
a notched joint positioned along the post between the first and second ends, the notched joint having a smaller cross-sectional area than adjacent portions of the first and second sections to form a weakened section that fractures upon the application of force to remove a distal section of the post between the notched joint and the second end from a remainder of the post.

9. The implant of claim 8, further comprising at least one additional joint along the post that is spaced away from the notched joint.

10. The implant of claim 8, wherein the notched joint includes a notch that extends around an entire periphery of the post.

11. The implant of claim 8, wherein the distal section of the post is constructed of a different material than the remainder of the post.

12. The implant of claim 8, wherein the remainder of the post includes a cross-sectional shape with at least one flat side.

13. The implant of claim 8, wherein the notched joint is positioned in closer proximity to the second end of the post than to the first end.

14. A method of attaching an implant to spinous processes comprising:
positioning a first plate on a first lateral side of the spinous processes;
positioning a post that extends outward from the first plate through an interspinous space formed between the spinous processes;
positioning a second plate on a second lateral side of the spinous processes with the post extending through a bore in the second plate;
sliding the second plate along the post and towards the spinous processes from a distal section of the post, over a joint, and onto a proximal section of the post;
positioning the first plate against the first lateral side of the spinous processes and the second plate against the second lateral side of the spinous processes;
securing the second plate on the proximal section of the post with the first and second plates contacting against the lateral sides of the spinous processes; and
detaching the distal section of the post from the proximal section of the post at the joint.

15. The method of claim 14, further comprising moving the plates against the lateral sides of the spinous processes and applying a compressive force to the spinous processes.

16. The method of claim 14, wherein detaching the distal section of the post from the proximal section of the post at the joint comprises unscrewing the distal section from the proximal section.

17. The method of claim 14, wherein joint includes a notch and has a smaller cross sectional size than the proximal and distal sections and detaching the distal section of the post from the proximal section of the post at the joint comprises applying a force to the distal section and fracturing the joint.

18. The method of claim 14, wherein detaching the distal section of the post from the proximal section of the post at the joint comprises applying a force to the distal section and rupturing a void in an interior of the post at the joint.

19. The method of claim 14, wherein detaching the distal section of the post from the proximal section of the post at the joint comprises fracturing an abutment face between two different materials.

20. The method of claim 14, further sliding the second plate along the post and towards the spinous processes from the distal section of the post, over a second joint that is spaced apart from the joint, over an intermediate section between the second joint and the joint, over the joint, and onto the proximal section of the post.
